(19) United States
(12) Patent Application Publication
(10) Pub. No.: US 2025/0032500 A1
(43) Pub. Date: Jan. 30, 2025

(54) REVITALIZING EYE DROPS

(71) Applicant: Revitalize Energy, Inc., Pittsburgh, PA (US)

(72) Inventor: Kyle Guinness, Pittsburgh, PA (US)

(21) Appl. No.: 18/782,664

(22) Filed: Jul. 24, 2024

Related U.S. Application Data
(60) Provisional application No. 63/515,277, filed on Jul. 24, 2023.

(51) Int. Cl. A61K 31/522; A61K 9/00; A61K 31/4164; A61K 33/30; A61K 47/10; A61K 47/18; A61K 47/20; A61K 47/38

(52) U.S. Cl. CPC A61K 31/522; A61K 9/0048; A61K 31/4164; A61K 33/30; A61K 47/10; A61K 47/183; A61K 47/20; A61K 47/38

(57) ABSTRACT

An ophthalmic composition for tired eye therapy comprising effective amounts of at least one or more stimulants, one or more non-essential amino acids, or one or more cooling agents. Additional compounds for the ophthalmic composition as well as methods of making and using the ophthalmic compositions are further disclosed.

### CROSS-REFERENCE TO RELATED APPLICATIONS

This application claims the priority benefit of U.S. Provisional Patent Application Ser. No. 63/515,277, filed Jul. 24, 2023, which is hereby incorporated herein by reference.

### BACKGROUND

Eyestrain, or asthenopia, is the condition in which the eyes become fatigued. Common causes include too much time spent looking at screens and lack of sleep. The result is often ophthalmic discomfort, which includes but is not limited to, dry, watery, burning, or itchy eyes. The present treatment for this affliction is often over the counter (“OTC”) artificial tears, wherein the mode of lubrication is through the use of viscosity enhancing agents or emulsions. While this treatment has been shown to help alleviate some of the symptoms associated with tired eyes, an ophthalmic drug that is formulated to have a more dramatic improvement on this very prevalent condition would be very desirable.

Stimulants work by promoting brain activity and when ingested, can increase a sense of awakeness and energy. It has presently been studied that the topical use of an ophthalmic drug consisting of a stimulant, such as caffeine, can prove to have a similar effect when applied to the eyes and be very effective in treating asthenopia. It has also been shown that in some cases, certain stimulants can treat cataracts.

While there are topical ophthalmic drugs which aim to address and prevent the ophthalmic irritants associated with asthenopia, there does not exist a drug with the present invention's unique combination of ingredients. While there are a few topical ophthalmic drugs available that claim to treat cataracts, there are not any topical ophthalmic drugs that utilize a stimulant.

### SUMMARY OF THE INVENTION

Although the present invention has been described in considerable detail with reference to certain preferred embodiments thereof, other versions are possible. Therefore, the spirit and scope of the appended claims should not be limited to the description and the preferred versions contained within this specification. Various aspects of the present invention will be illustrated with reference to the following non-limiting examples.

### DETAILED DESCRIPTION

It should be stated that the terminology used is not meant to limit any possible embodiments and the present invention is not limited in scope by the described compositions or suggested methods of use. Additional active and inactive ingredients can be included in various embodiments if they are within their designated therapeutic ranges and do not have adverse effects on the other ingredients.

The term “about” as used herein in reference to the suggested concentration of ingredients should be understood to mean ±10% of the value.

The term “therapeutic concentration” or “therapeutic amount” as used herein is meant to be interpreted as the amount of a certain ingredient at which the desired effect will be achieved on the body.

The phrase “preferred concentration” should be read with “therapeutic concentration” in mind, as it is used to refer to what is desired based on what is proven to be effective.

The term “treat” or variations thereof used in this context can mean, but is not limited to, the alleviation of symptoms, improving the current state of a condition, or preventing a worsening state of a condition or symptoms associated.

Various embodiments described herein are directed to ophthalmic compositions containing a therapeutically effective amount of one or more stimulants. For example, certain embodiments described herein are directed to ophthalmic compositions containing caffeine. Further embodiments are directed to methods for treating maladies of the eye including, for example, tired eyes, ophthalmic discomfort, cataracts, conjunctivitis, and the like and combinations thereof, that include topically administering an effective amount of the ophthalmic composition to the eyes of a subject in need of treatment, for example, by means of eye drops. Ophthalmic discomfort can be caused by many issues including eye fatigue and tiredness, surgery, and diseases of the eye. Examples of ophthalmic discomfort can include watery eyes, sore or tired eyes, dry eyes, blurred vision, double vision, sensitivity to light, headache, neck and shoulder pain, difficulty concentrating, burning eyes, itchy eyes, and difficulty keeping the eyes open.

In certain embodiments, the ophthalmic compositions described herein can further include one or more non-essential amino acids, one or more cooling agents, or combinations thereof. For example, in some embodiments, the ophthalmic compositions can include one or more stimulants and one or more non-essential amino acid while in other embodiments, the ophthalmic compositions can include one or more stimulants and one or more cooling agents. In certain embodiments, the ophthalmic compositions can include one or more stimulants, one or more non-essential amino acids, and one or more cooling agents.

In yet other embodiments, the ophthalmic compositions can alternatively include at least one of the one or more stimulants, one or more non-essential amino acids, and one or more cooling agents. For example, in certain embodiments, an ophthalmic composition can include one or more non-essential amino acids or can include one or non-essential amino acids and one or more cooling agents. Certain such embodiments can be free of any stimulants and, in such embodiments, the ophthalmic discomfort can be treated by the non-essential amino acids and/or cooling agents.

Suitable compounds which can act as a stimulant in various embodiments include, for example, caffeine, ephedrine, pseudoephedrine, citrulline malate, green tea extract, branched chain amino acids, magnesium-containing compounds, and the like and combinations thereof. In particular embodiments, the stimulant can be caffeine. The amount of stimulant can vary among embodiments and can be any therapeutically effective concentration. For example, in some embodiments, the concentration of the stimulant in the ophthalmic compositions described herein can be about 0.01% (w/w) to about 10% (w/w), about 0.02% (w/w) to about 9.5% (w/w), about 0.03% (w/w) to about 9.0% (w/w), about 0.04% (w/w) to about 8.5% (w/w), about 0.05% (w/w) to about 8.0% (w/w), about 0.06% (w/w) to about 7.5% (w/w), about 0.07% (w/w) to about 7.0% (w/w), about 0.08% (w/w) to about 6.5% (w/w), about 0.1% (w/w) to about 6.0% (w/w), about 0.25% (w/w) to about 5.5% (w/w), about 0.5% (w/w) to about 5.0% (w/w), about 0.75% (w/w) to about 3% (w/w), about 1.0% (w/w) to about 3.0% (w/w), or any individual concentration or range encompassed by these example ranges.

Non-essential amino acids, like taurine, can act as an anti-inflammatory when applied ophthalmically and may treat cataracts caused by hyperglycemia. The benefits of non-essential amino acids in an ophthalmic composition can be enhanced through combination with stimulants and cooling agents. For example, the combination of non-essential amino acids with stimulants, like caffeine, can further improve the effect of the non-essential amino acids and further revitalize the eye. Addition of a cooling agent can provide a cooling sensation to the eyes and may increase tear production and provide lubrication to the eyes. As can be appreciated, eyes can exhibit reduced tear production as a result of injury, fatigue, or disease and require improved lubrication which can be stimulated by the cooling agent. Thus, the ophthalmic compositions of the present disclosure provide beneficial effects by combining, for example, caffeine, taurine, and menthol together to treat injured, fatigued, or diseased eyes.

The non-essential amino acids used in various embodiments can include for example, glycine, alanine, proline, serine, glutamic acid, aspartic acid, asparagines, taurine, carnitine, and the like and combinations thereof. In particular embodiments, the non-essential amino acid can be taurine. The amount of non-essential amino acids can vary among embodiments and can be any therapeutically effective concentration. For example, in some embodiments, the amount of non-essential amino acids in the ophthalmic compositions described herein can be about 0.01% (w/w) to about 10% (w/w), about 0.02% (w/w) to about 9.5% (w/w), about 0.03% (w/w) to about 9.0% (w/w), about 0.04% (w/w) to about 8.5% (w/w), about 0.05% (w/w) to about 8.0% (w/w), about 0.06% (w/w) to about 7.5% (w/w), about 0.07% (w/w) to about 7.0% (w/w), about 0.08% (w/w) to about 6.5% (w/w), about 0.1% (w/w) to about 6.0% (w/w), about 0.25% (w/w) to about 5.5% (w/w), about 0.5% (w/w) to about 5.0% (w/w), about 0.75% (w/w) to about 4.5% (w/w), about 1.0% (w/w) to about 3.0% (w/w), or any individual concentration or range encompassed by these example ranges.

In some embodiments, the ophthalmic composition can include a cooling agent. The cooling agent can be any compound that has the ability to trigger certain receptors in the body which result in a sensation of coolness such as, for example, menthol, borneol, isopulegol, 3-(1-menthoxy) propan-1,2-diol, 3-(1-menthoxy)-2-methylpropan-1,2-diol, p-menthan-2,3-diol, p-menthan-3,8-diol, 6-isopropyl-9-methyl-1,4-dioxaspiro[4,5]decan-2-methanol, menthyl succinate and its alkaline earth metal salts, trimethylcyclohexanol, N-ethyl-2-isopropyl-5-methylcyclohexanecarboxamide, peppermint oil, peppermint oil, menton, menthol glycerol ketal, menthyl lactate, 3-(1-menthoxy) ethan-1-ol, 3-(1-menthoxy) propan-1-ol, 3-(1-methoxy) butan-1-ol, 1-methyl-acetic acid N-ethylamide, 1-methyl-4-hydroxypentanoate, 1-methyl-3-hydroxybutyrate, N-2,3-trimethyl-2-(1-methylethyl) butanamide, n-ethyl-t-2-s-6-nonadienamide, N,N-dimethylmethyl succinamide, methyl pyrrolidone carboxylate, and the like and combinations thereof. In particular embodiments, the cooling agent can be one or more of menthol and borneol. In certain embodiments, the cooling agent can be one or more of L-menthol and D-borneol. The amount of the cooling agent thereof contained in the ophthalmic compositions can be any therapeutically effective concentration. For example, a therapeutically effective amount of menthol in ophthalmic compositions of the present disclosure can be about 0.005% (w/w) to about 0.1% (w/w), about 0.01% (w/w) to about 0.09% (w/w), about 0.02% (w/w) to about 0.08% (w/w), about 0.03% (w/w) to about 0.07% (w/w), about 0.04% (w/w) to about 0.06% (w/w), or any individual concentration or range encompassed by these example ranges.

In some embodiments, the ophthalmic compositions can further include an ophthalmic astringent. Examples of suitable astringents can include, but are not limited to, zinc sulfate, tetrahydrozoline, and the like and combinations thereof. An ophthalmic astringent can constrict eye tissue and eliminate ophthalmic discomfort. The concentration of the ophthalmic astringent can vary among embodiments and can be about 0.005% (w/w) to about 0.1% (w/w), about 0.01% (w/w) to about 0.09% (w/w), about 0.02% (w/w) to about 0.08% (w/w), about 0.03% (w/w) to about 0.07% (w/w), about 0.04% (w/w) to about 0.06% (w/w), about 0.05% (w/w) to about 5% (w/w) or any individual concentration or range encompassed by these example ranges.

In some embodiments, the ophthalmic compositions can include a viscosity enhancing agent, which can lubricate the surface of the eye. Examples of suitable viscosity agents that can be used in such ophthalmic compositions include, but are not limited to, hydroxypropyl methylcellulose (“hypromellose”), polyvinyl alcohol, polyvinyl pyrrolidone, methyl cellulose, hydroxyethyl cellulose, carboxymethyl cellulose, and hydroxypropyl cellulose. The concentration of the viscosity enhancing agent can vary among embodiments and can be about 0.005% (w/w) to about 0.1% (w/w), about 0.01% (w/w) to about 0.09% (w/w), about 0.02% (w/w) to about 0.08% (w/w), about 0.03% (w/w) to about 0.07% (w/w), about 0.04% (w/w) to about 0.06% (w/w), about 0.01% (w/w) to about 2% (w/w) or any individual concentration or range encompassed by these example ranges.

In some embodiments, the ophthalmic compositions can include solvents, inert diluents, buffering agents, dispersing or wetting agents, gelling agents, preservatives, chelating agents, anti-foaming agents, antioxidants, permeation enhancers, tonicity agents, additional amino acids, ophthalmic vasoconstrictors, solubilizers, antimicrobial agents, anti-inflammatoires, steroids, anesthetics, demulcents, vitamins, and the like and combinations thereof.

Naturally occurring tears commonly contain amino acids and inclusion of such amino acids in the ophthalmic compositions described herein can be useful. Thus, the ophthalmic compositions described herein may include amino acids other than the non-essential amino acids described above. For example, in some embodiments, the ophthalmic compositions can include amino acids such as, but not limited to, γ-aminobutyric acid, alanine, cysteine, serine, taurine, threonine, valine, histidine, 4-hydroxyproline, phenylalanine, proline, L-aspartate, ¿-aminocaproic acid, lysine, arginine, salts thereof, and the like and combinations thereof. For example, in certain embodiments, the potassium salt of L-aspartate, potassium L-aspartate can be included in an ophthalmic composition described herein. The concentration of amino acids in the ophthalmic compositions can vary among embodiments and can be about 0.005% (w/w) to about 5% (w/w), about 0.01% (w/w) to about 3.0% (w/w), about 0.02% (w/w) to about 1.5% (w/w), or any individual concentration or range encompassed by these example ranges.

In some embodiments, the ophthalmic compositions can include vasoconstrictors, which may reduce redness and alleviate some ophthalmic discomfort by narrowing the blood vessels in the eye. Examples of suitable vasoconstrictors for the ophthalmic compositions can include, but are not limited to, tetrahydrozoline, naphazoline, phenylephrine, salts thereof, and combinations thereof. For example, in certain embodiments, a suitable vasoconstrictor can be the naphazoline salt, naphazoline hydrochloride. The concentration of vasoconstrictors in the ophthalmic compositions can vary among embodiments and can be about 0.005% (w/w) to about 1% (w/w), about 0.01% (w/w) to about 0.9% (w/w), about 0.02% (w/w) to about 0.8% (w/w), about 0.025% (w/w) to about 0.7% (w/w), about 0.03% (w/w) to about 0.6% (w/w), or any individual concentration or range encompassed by these example ranges.

In some embodiments, the ophthalmic compositions can include various surface modifying compounds such as surfactants, lubricants, and solubilizers. Examples of such components can include, but are not limited to, polysorbate 80, tyloxapol, polyoxyethylene, fatty acid glycerol polyethylene glycol esters, fatty acid polyethylene glycol esters, propylene glycol, polyethylene glycols, glycerol ethers, butylene glycol (e.g., Brontide®), cyclodextrin, for example, alpha-, beta- or gamma-cyclodextrin, alkylated, hydroxyalkylated, carboxyalkylated or alkyloxycarbonyl-alkylated derivatives, or mono- or diglycosyl-alpha-, beta- or gamma-cyclodextrin, mono- or dimaltosyl-alpha-, beta- or gamma-cyclodextrin or panosyl-cyclodextrin, polysorbate 20, and the like and combinations or mixtures thereof. The concentration of such surface modifying compounds in the ophthalmic compositions can vary among embodiments and can be about 0.1% (w/w) to about 15% (w/w), about 0.5% (w/w) to about 10% (w/w), about 0.75% (w/w) to about 5% (w/w), about 1.0% (w/w) to about 2% (w/w), or any individual concentration or range encompassed by these example ranges.

In order to stabilize the pH of the ophthalmic compositions, some embodiments may include buffers such as, but not limited to, citric acid, boric acid, acetic acid, tartaric acid, and the like and combinations thereof. The buffers can be provided in any amount sufficient to adjust and buffer the ophthalmic composition to a pH of about 5.0 to about 8.0 and to an osmolarity of about 200 mOsmol/kg to about 450 mOsmol/kg.

In some embodiments, the ophthalmic compositions can include an antimicrobial agent. Examples of suitable antimicrobial agents can include, but are not limited to, benzalkonium chloride, thimerosal, chlorobutanol, methyl paraben, propylparaben, phenylethyl alcohol, ethylenediaminetetraacetic acid (“EDTA”), sorbic acid, Onamer® M (polyquaternium-1), sodium chloride, tyloxapol, sodium sulfate, hydroxyethylcellulose, silver sulfadiazine, colloidal silver, hydrogen peroxide, polyhexamethylene biguanide, myristamidopropyl dimethylamine, boric acid, and the like and combinations thereof. The concentration of the antimicrobial agents in can vary among embodiments and can be about 0.001% (w/w) to about 1% (w/w), about 0.005% (w/w) to about 0.5% (w/w), about 0.01% (w/w) to about 0.1% (w/w) or any individual concentration or range encompassed by these example ranges. The antimicrobial agent can also act as a preservative.

In some embodiments, the ophthalmic compositions can include an anti-inflammatory agent. Examples of suitable anti-inflammatory agents can include, but are not limited to, camphor, ketotifen fumarate, diclofenac sodium, flurbiprofen sodium, ketorolac tromethamine, suprofen, celecoxib, naproxen, rofecoxib, and the like and combinations thereof. The concentration of anti-inflammatory in the ophthalmic compositions can vary among embodiments and can be about 0.005% (w/w) to about 5% (w/w), about 0.01% (w/w) to about 1% (w/w), about 0.02% (w/w) to about 0.5% (w/w), or any individual concentration or range encompassed by these example ranges.

In some embodiments, the ophthalmic compositions can include a steroid. Examples of suitable steroids that can be used in various embodiments include, but are not limited to, dexamethasone, dexamethasone alcohol, dexamethasone sodium phosphate, fluorometholone acetate, fluorometholone alcohol, loteprednol etabonate, medrysone, prednisolone, prednisone, prednisolone acetate, prednisolone sodium phosphate, rimexolone, hydrocortisone, hydrocortisone acetate, lodoxamide tromethamine, methylprednisolone, and the like and combinations thereof. The concentration of steroid in the ophthalmic compositions can vary among embodiments and can be about 0.05% (w/w) to about 5% (w/w), about 0.1% (w/w) to about 4% (w/w), about 1% (w/w) to about 3% (w/w), about 0.5% (w/w) to about 3% (w/w), or any individual concentration or range encompassed by these example ranges.

Some embodiments of the ophthalmic composition can include an anesthetic such as, for example, proparacaine, lidocaine tetracaine, and the like and combinations thereof. The concentration of anesthetic in the ophthalmic compositions can vary among embodiments and can be about 0.05% (w/w) to about 5% (w/w), about 0.1% (w/w) to about 4% (w/w), about 1% (w/w) to about 3% (w/w), about 0.5% (w/w) to about 3% (w/w), or any individual concentration or range encompassed by these example ranges.

In some embodiments, the ophthalmic compositions can include a demulcent. Examples of suitable demulcents include, but are not limited to, cellulose derivatives, carboxymethylcellulose sodium, hydroxyethyl cellulose, hydroxypropyl methylcellulose, methylcellulose, dextran 70, gelatin, liquid polyols, glycerin, polyethylene glycol 300, polyethylene glycol 400, polysorbate 80, polyvinyl alcohol, povidone, and the like and combinations thereof. The concentration of demulcent in the ophthalmic compositions can vary among embodiments and may be is about 0.05% (w/w) to about 5% (w/w), about 0.1% (w/w) to about 4% (w/w), about 1% (w/w) to about 3% (w/w), about 0.5% (w/w) to about 3% (w/w), or any individual concentration or range encompassed by these example ranges.

In some embodiments, the ophthalmic compositions can include a vitamin such as, but not limited to, include vitamin A, vitamin B5, vitamin B12, vitamin C, vitamin D and the like and combinations thereof. The concentration of vitamins in the ophthalmic compositions can vary among embodiments and may be is about 0.05% (w/w) to about 5% (w/w), about 0.1% (w/w) to about 4% (w/w), about 1% (w/w) to about 3% (w/w), about 0.5% (w/w) to about 3% (w/w), or any individual concentration or range encompassed by these example ranges.

As can be appreciated, certain compounds can exhibit multiple effects when included in an ophthalmic composition. Although certain components may be listed as providing a particular effect, other benefits can also be provided by the same component and could serve other functions in the ophthalmologic eye drops disclosed herein. For example, camphor can provide both a cooling sensation as well as an anti-inflammatory effect while boric acid can be used to provide both pH buffering as well as anti-septic effects.

In certain embodiments, the ophthalmic compositions disclosed herein can include enantiomeric compounds such as borneol, camphor, and amino acids. Such compounds can be used in a particular absolute configuration, such as D-borneol or D-camphor, while in other embodiments, any form of the compound can be used including racemic versions such as DL-camphor.

In particular embodiments, the ophthalmic composition can include caffeine, taurine, zinc sulfate, hypromellose, and menthol in sufficient quantities to provide an eye drop that can treat eye fatigue, eye injury, or eye disease. For example, in certain embodiments, the ophthalmic compositions can include about 0.01% to about 10% (w/w) caffeine, about 0.1 to about 3.0% (w/w) taurine, about 0.05 to about 5% (w/w) zinc sulfate, about 0.01 to about 2% (w/w) hypromellose, and about 0.005 to about 0.1% (w/w) menthol. In other example embodiments, the ophthalmic composition can include about 0.01% (w/w) to about 10% (w/w) caffeine, about 0.1% (w/w) to about 3.0% (w/w) taurine, and about 0.01% (w/w) to about 1% (w/w) menthol. All such example compositions can further include additional components such as a lubricating agent, additional amino acids, pH stabilizers, anti-inflammatory agents, and preservatives as well as a solvent such as water, saline, or dilute alcohol. For example, an ophthalmic composition according to some embodiments can include about 0.01% (w/w) to about 10% (w/w) caffeine, about 0.1% (w/w) to about 3.0% (w/w) taurine, about 0.01% (w/w) to about 1% (w/w) menthol, about 0.001% to about 0.1% (w/w) naphazoline hydrochloride, about 0.5% to about 5% (w/w) propylene glycol, about 0.001% to about 0.2% (w/w) borneol, about 0.005% to about 0.2% (w/w) camphor, about 0.1% to about 5% (w/w) potassium L-aspartate, about 0.001% to about 2% (w/w) benzalkonium chloride, about 0.1% to about 2% (w/w) boric acid, and the remainder water.

Additional embodiments are directed to methods for treating eye irritation, fatigue, disease, ophthalmic discomfort, and the like and combinations thereof by administering an ophthalmic composition as described herein. The disclosed compositions may be used to treat, prevent, or reduce the intensity of one or more symptoms of eye fatigue or eye strain selected from watery eyes, sore or tired eyes, dry eyes, blurred vision, double vision, sensitivity to light, headache, neck and shoulder pain, difficulty concentrating, burning eyes, itchy eyes, and difficulty keeping the eyes open. In some embodiments, the ophthalmic compositions may include at least one of one or more stimulants, one or more non-essential amino acids, one or more cooling agents. The stimulants, non-essential amino acids, and cooling agents can be any of the compounds identified above in any of the concentrations discussed in relation to these ingredients. In some embodiments, the ophthalmic compositions may include any of the solvents, inert diluents, buffering agents, dispersing or wetting agents, gelling agents, preservatives, chelating agents, anti-foaming agents, antioxidants, gelling agents, permeation enhancers, tonicity agents, additional amino acids, ophthalmic vasoconstrictors, solubilizers, antimicrobial agents, anti-inflammatoi steroids, anesthetics, demulcents, vitamins, and the like and combinations thereof described above in quantities identified.

The step of administering can be carried out in any effective way. For example, in some embodiments, administering can include topically applying the ophthalmic composition to the eye in drops. Each eye may receive from 1 to 10 or more drops or any amount sufficient to wet and lubricate the eye while providing relief to the subject in need of treatment. In some embodiments, administering can include topically applying the ophthalmic composition to the eye as a mist that is sprayed on to the eye, and in certain embodiments, administering may include washing the eye with the ophthalmic composition such that the eye and surrounding tissues are wetted.

Administering can be carried out at the discretion of the subject. For example, in some embodiments, administering can be carried out once, twice, three times, or 4, 5, 6, 7, 8, 9, or 10 times per day. In certain embodiments, administering can be carried out at least once or at least twice per day until the subject experiences relief from the symptoms of the underlying eye irritation, eye fatigue, eye disease, and the like.

Generally, the ophthalmic compositions disclosed herein can be formed using methods known in the art. For example, each of the components can be mixed together and then packaged into a sterile eye-dropper bottle. In certain embodiments, each of the components can be mixed together and then adjusted for pH. Anti-tampering and safety features such as seals, dates of manufacture, and expiration dates can be applied to the bottle.

The ophthalmic compositions of various embodiments may provide relief from eye irritation, eye fatigue, dry eye, eye disease, and the like and chronic forms of these maladies by lubricating and cooling the eye while stimulating the eye and surrounding tissues. Lubricating and cooling the eye provides relief from symptoms related to eye irritation, eye fatigue, eye disease, and the like improving the subjects vision and alertness.

### EXAMPLES

Although the present invention has been described in considerable detail with reference to certain preferred embodiments thereof, other versions are possible. Therefore, the spirit and scope of the appended claims should not be limited to the description and the preferred versions described within this specification. Various aspects of the present invention will be illustrated with reference to the following non-limiting examples.

### Example 1

An eye drop was prepared using 0.200% (w/w) hypromellose, 0.050% (w/w) tetrahydrozoline hydrochloride, 0.250% (w/w) zinc sulfate, 97.783% (w/w) purified water, 0.035% (w/w) boric acid, 0.280% (w/w) sodium borate, 0.200% (w/w) polysorbate 80, 0.002% (w/w) menthol, 0.200% (w/w) brontide, 0.500% (w/w) caffeine, and 0.500% (w/w) taurine. The pH was adjusted using diluted citric acid.

### Example 2

An eye drop was prepared using 0.200% (w/w) hypromellose, 0.050% (w/w) tetrahydrozoline hydrochloride, 0.250% (w/w) zinc sulfate, 97.781% (w/w) purified water, 0.035% (w/w) boric acid, 0.280% (w/w) sodium borate, 0.200% (w/w) polysorbate 80, 0.004% (w/w) menthol, 0.200% (w/w) brontide, 0.500% (w/w) caffeine, and 0.500% (w/w) taurine. The pH was adjusted using diluted citric acid.

### Example 3

An eye drop was prepared using 0.200% (w/w) hypromellose, 0.050% (w/w) tetrahydrozoline hydrochloride, 0.250% (w/w) zinc sulfate, 97.777% (w/w) purified water, 0.035% (w/w) boric acid, 0.280% (w/w) sodium borate, 0.200% (w/w) polysorbate 80, 0.008% (w/w) menthol, 0.200% (w/w) brontide, 0.500% (w/w) caffeine, and 0.500% (w/w) taurine. The pH was adjusted using diluted citric acid.

### Example 4

An eye drop was prepared using 0.030% (w/w) naphazoline hydrochloride, 1.000% (w/w) propylene glycol, 0.011% (w/w) D-borneol, 0.022% (w/w) DL-camphor, 0.035% (w/w) L-menthol, 1.000% (w/w) caffeine, 1.000% (w/w) potassium L-aspartate, 1.000% (w/w) taurine, 0.010% (w/w) benzalkonium chloride (50% active), 0.530% (w/w) boric acid, and 95.362% (w/w) purified water. The pH was adjusted using diluted citric acid.

For each of Examples 1 to 4, the eye drops prepared were found to effectively treat and alleviate symptoms of ophthalmic discomfort caused by eye fatigue and tiredness as well as the sensation of dry eyes. Each of Examples 1-3 were found to be stable for a duration of at least one month. Example 4 was found to be stable for a duration of 3 months when evaluated at temperatures of 25° C., 30° C., and 35° C.

What is claimed is:

1. A topical ophthalmic composition that treats ophthalmic discomfort or tired eyes, the topical ophthalmic composition comprising:
one or more stimulants; and
one or more additional components, the one or more additional components comprising one or more non-essential amino acids and one or more cooling agents.

2. The topical ophthalmic composition of claim 1, comprising an effective amount of the one or more stimulants.

3. The topical ophthalmic composition of claim 1, wherein the one or more stimulants comprise caffeine.

4. The topical ophthalmic composition of claim 3 comprises about 0.01 to about 10% (w/w) caffeine.

5. The topical ophthalmic composition of claim 1 comprising one or more non-essential amino acids, wherein the one or more non-essential amino acids comprise glycine, alanine, proline, serine, glutamic acid, aspartic acid, asparagine, taurine, or carnitine.

6. The topical ophthalmic composition of claim 5 comprises about 0.1 to about 3.0% (w/w) taurine.

7. The topical ophthalmic composition of claim 1 comprising one or more cooling agents, wherein the one or more cooling agents comprise menthol, borneol, isopulegol, 3-(1-menthoxy) propan-1,2-diol, 3-(1-menthoxy)-2-methylpropan-1,2-diol, p-menthan-2,3-diol, p-menthan-3,8-diol, 6-isopropyl-9-methyl-1,4-dioxaspiro[4,5]decan-2-methanol, menthyl succinate and its alkaline earth metal salts, trimethylcyclohexanol, N-ethyl-2-isopropyl-5-methylcyclohexanecarboxamide, peppermint oil, peppermint oil, menton, menthol glycerol ketal, menthyl lactate, 3-(1-menthoxy) ethan-1-ol, 3-(1-menthoxy) propan-1-ol, 3-(1-methoxy) butan-1-ol, 1-methyl-acetic acid N-ethylamide, 1-methyl-4-hydroxypentanoate, 1-methyl-3-hydroxybutyrate, N-2,3-trimethyl-2-(1-methylethyl) butanamide, n-ethyl-t-2-s-6-nonadienamide, N,N-dimethylmethyl succinamide, or methyl pyrrolidone carboxylate.

8. The topical ophthalmic composition of claim 7 comprises about 0.005-0.1% (w/w) menthol.

9. The topical ophthalmic composition of claim 1 comprises menthol and taurine.

10. The topical ophthalmic composition of claim 1, further comprising an effective amount of a viscosity enhancing agent.

11. The topical ophthalmic composition of claim 10, wherein the viscosity enhancing agent comprises one or more of hydroxypropyl methylcellulose, polyvinyl alcohol, polyvinyl pyrrolidone, methyl cellulose, hydroxyethyl cellulose, carboxymethyl cellulose, and hydroxypropyl cellulose.

12. The topical ophthalmic composition of claim 1, further comprising an effective amount of an ophthalmic astringent.

13. The topical ophthalmic composition of claim 12, wherein the ophthalmic astringent comprises one or more of zinc sulfate and tetrahydrozoline.

14. The topical ophthalmic composition of claim 1, further comprising one or more of solvents, viscosity enhancing agents, inert diluents, buffering agents, dispersing or wetting agents, gelling agents, preservatives, chelating agents, anti-foaming agents, antioxidants, permeating enhancers, tonicity agents, additional amino acids, ophthalmic vasoconstrictors, solubilizers, antimicrobial agents, anti-inflammatoires, steroids, anesthetics, demulcents, and vitamins.

15. The topical ophthalmic composition of claim 14, wherein the viscosity enhancing agents comprises one or more of hydroxypropyl methylcellulose (“hypromellose”), polyvinyl alcohol, polyvinyl pyrrolidone, methyl cellulose, hydroxyethyl cellulose, carboxymethyl cellulose, and hydroxypropyl cellulose.

16. The topical ophthalmic composition of claim 1, further comprising a pH buffer comprising one or more of citric acid, boric acid, acetic acid, tartaric acid; and
wherein the ophthalmic composition comprises a pH of about 5.0 to about 8.0 and an osmolarity of about 200 mOsmol/kg to about 450 mOsmol/kg.

17. A topical ophthalmic composition comprising (w/w):
about 0.01% to about 10% of a stimulant;
about 0.005% to about 0.1% of a cooling agent;
about 0.1% to about 3% of aa non-essential amino acid;
about 0.05% to about 5% of an ophthalmic astringent; and
about 0.01% to about 2% of a viscosity enhancing agent.

18. The topical ophthalmic composition of claim 17, wherein:
the stimulant comprises caffeine;
the cooling agent comprises menthol;
the non-essential amino acid comprises taurine;
the ophthalmic astringent comprises zinc sulfate; and
the viscosity enhancing agent comprises hypromellose.

19. A method of treating one or more eye conditions comprising the steps of:
administering a therapeutically effective amount of a topical ophthalmic composition to a patient in need; and
wherein the one or more eye conditions comprise tired eyes, ophthalmic discomfort, cataracts, and conjunctivitis.

20. The method of claim 19, wherein the step of administering the therapeutically effective amount of the topical ophthalmic composition alleviates one or more symptoms including watery eyes, sore or tired eyes, dry eyes, blurred vision, double vision, sensitivity to light, headache, neck and shoulder pain, difficulty concentrating, burning eyes, itchy eyes, and difficulty keeping the eyes open.
